(19) United States
(12) Patent Application Publication
(10) Pub. No.: US 2005/0085818 A1
(43) Pub. Date: Apr. 21, 2005

(54) Systems for distal radius fixation

(76) Inventor: Randall J. Huebner, Beaverton, OR (US)

Correspondence Address: KOLISCH HARTWELL, P.C., 520 S.W. YAMHILL STREET, SUITE 200, PORTLAND, OR, 97204, US

(21) Appl. No.: 10/968,850

(22) Filed: Oct. 18, 2004

Related U.S. Application Data
(60) Provisional application No. 60/512,111, filed on Oct. 17, 2003.

(51) Int. Cl. A61B 17/58

(52) U.S. Cl. 606/69

(57) ABSTRACT

Systems, including methods, apparatus, and kits, for fixing the distal radius with bone plates.

### CROSS-REFERENCE TO PRIORITY APPLICATION

This application is based upon and claims the benefit under 35 U.S.C. § 119(e) of U.S. Provisional Patent Application Ser. No. 60/512,111, filed Oct. 17, 2003, which is incorporated herein by reference in its entirety for all purposes.

### CROSS-REFERENCES TO RELATED MATERIALS

This application incorporates by reference the following U.S. patents: U.S. Pat. No. 6,030,162, issued Dec. 18, 1998; and U.S. Pat. No. 6,299,615, issued Aug. 16, 1999.

This application also incorporates by reference the following U.S. patent applications: Ser. No. 10/625,503, filed Jul. 22, 2003; Ser. No. 10/716,719, filed Nov. 19, 2003; Ser. No. 10/717,015, filed Nov. 19, 2003; Serial No. 717,399, filed Nov. 19, 2003; Ser. No. 10/717,401, filed Nov. 19, 2003; Ser. No. 10/717,402, filed Nov. 19, 2003; Ser. No. 10/731,173, filed Dec. 8, 2003; Ser. No. 10/873,522, filed Jun. 21, 2004; Ser. No. 10/873,410, filed Jun. 21, 2004; and Ser. No. 10/927,824, filed Aug. 27, 2004.

This application also incorporates by reference the following U.S. provisional patent applications: Ser. No. 60/541,414, filed Feb. 2, 2004; Ser. No. 60/548,685, filed Feb. 26, 2004; Ser. No. 60/563,767, filed Apr. 19, 2004; Ser. No. 60/563,860, filed Apr. 19, 2004; and Ser. No. 60/564,863, filed Apr. 22, 2004.

### BACKGROUND

The human skeleton is composed of 206 individual bones that perform a variety of important functions, including support, movement, protection, storage of minerals, and formation of blood cells. To ensure that the skeleton retains its ability to perform these functions, and to reduce pain and disfigurement, bones that become damaged should be repaired promptly and properly. Typically, a cut or fractured bone is treated using a fixation device, which reinforces the bone and keeps it aligned during healing. Fixation devices may include external fixation devices (such as casts or fixators) and/or internal fixation devices (such as bone plates and bone screws), among others.

Bone plates are sturdy internal devices, usually made of metal, that mount directly to the bone adjacent the fracture (or cut). These plates may be preformed and/or custom bent for mounting to particular portions of bone. To use a bone plate to repair a fracture of a bone, a surgeon (1) selects an appropriate plate, (2) reduces (sets) the fracture, and (3) fastens the plate to pieces of the bone on opposite sides of the fracture using suitable fasteners, such as screws and/or wires, so that the bone pieces are fixed in position.

The bones in the human skeleton can be grouped into two categories, the axial skeleton, and the appendicular skeleton. The axial skeleton includes, among others, the skull, vertebrae, and ribs. The appendicular skeleton includes, among others, the long bones of the upper and lower limbs, including the humerus, radius, and ulna. The radius is one of two long bones found in the human forearm. The radius, like other bones, is susceptible to a variety of fractures and other dislocations. For example, distal fractures of the radius are a common result, particularly among the elderly, of forward falls with the palms facing downward. A bone plate thus may be attached to a distal portion of the radius to fix the bone, but attachment may be problematic. For example, the radius of elderly patients often is formed of porous bone of poor quality. Accordingly, the bone plate may be secured with longer bone screws that extend completely through the radius, to improve purchase. However, these longer bone screws may extend into and irritate soft tissue. Furthermore, even with longer bone screws, one or more of the bone screws may work itself loose, so that the bone plate is no longer properly secured to the radius. Thus, there is a need for other approaches to securing a bone plate to the distal radius.

### SUMMARY

The present teachings provide systems, including methods, apparatus, and kits, for fixing the distal radius with bone plates.

### BRIEF DESCRIPTION OF THE DRAWINGS

FIG. 1 is a lateral view of a fractured distal radius of a right-sided appendage.

FIG. 2 is a lateral view of an exemplary system for fixation of the fractured distal radius of FIG. 1 using a bone plate and bone screws, including at least one bone screw locked to the bone plate at an angular offset, in accordance with aspects of the present teachings.

FIG. 3 is a sectional view of the system of FIG. 2, taken generally along line 3-3 of FIG. 2 and showing a bone screw locked at an angular offset, in accordance with aspects of the present teachings.

FIG. 4 is a sectional view of the system of FIG. 2, taken generally along line 4-4 of FIG. 2 and showing a bone screw locked orthogonally to the plate, in accordance with aspects of the present teachings.

FIG. 5 is a plan view of another exemplary bone plate configured to be secured to the distal radius and including a locking aperture configured to receive a bone screw that locks to the bone plate at an angular offset, in accordance with aspects of the present teachings.

FIG. 6 is a sectional view of the bone plate of FIG. 5, taken generally along line 6-6 of FIG. 5.

FIG. 7 is a sectional view of the bone plate of FIG. 5, taken generally along line 7-7 of FIG. 5.

FIG. 8 is a plan view of the bone plate of FIG. 5, secured to a volar surface of a fractured distal radius, with a plurality of fasteners locked to apertures of the bone plate, in accordance with aspects of the present teachings.

FIG. 9 is a perspective view of the bone plate and fasteners of FIG. 8, taken generally facing the inner surface of the bone plate in the absence of the fractured distal radius.

FIG. 10 is a fragmentary view of another exemplary bone plate for fixing distal radius fractures and including at least one locking aperture for receiving a bone screw at an angular offset, in accordance with aspects of the present teachings.

FIG. 11 is a fragmentary view of yet another exemplary bone plate for fixing distal radius fractures and including at least one locking aperture for receiving a bone screw at an angular offset, in accordance with aspects of the present teachings.

FIG. 12 is a view of an exemplary system for securing a bone plate to the distal radius, including an exemplary jig that attaches to the bone plate to guide formation of holes in bone and/or placement of fasteners into the bone, in accordance with aspects of the present teachings.

FIG. 13 is a view of the jig of FIG. 12 taken from above an outer surface of the jig.

FIG. 14 is a view of the jig of FIG. 12 taken from below an inner surface of the jig.

### DETAILED DESCRIPTION

The present teachings provide systems, including methods, apparatus, and kits, for fixing the distal radius with bone plates. The systems may include or use, among others, bone plates and fasteners (such as bone screws) that secure the bone plates to the distal radius. The systems also may include or use jigs configured to guide formation of holes and/or placement of fasteners into the distal radius, from or to apertures of the bone plates.

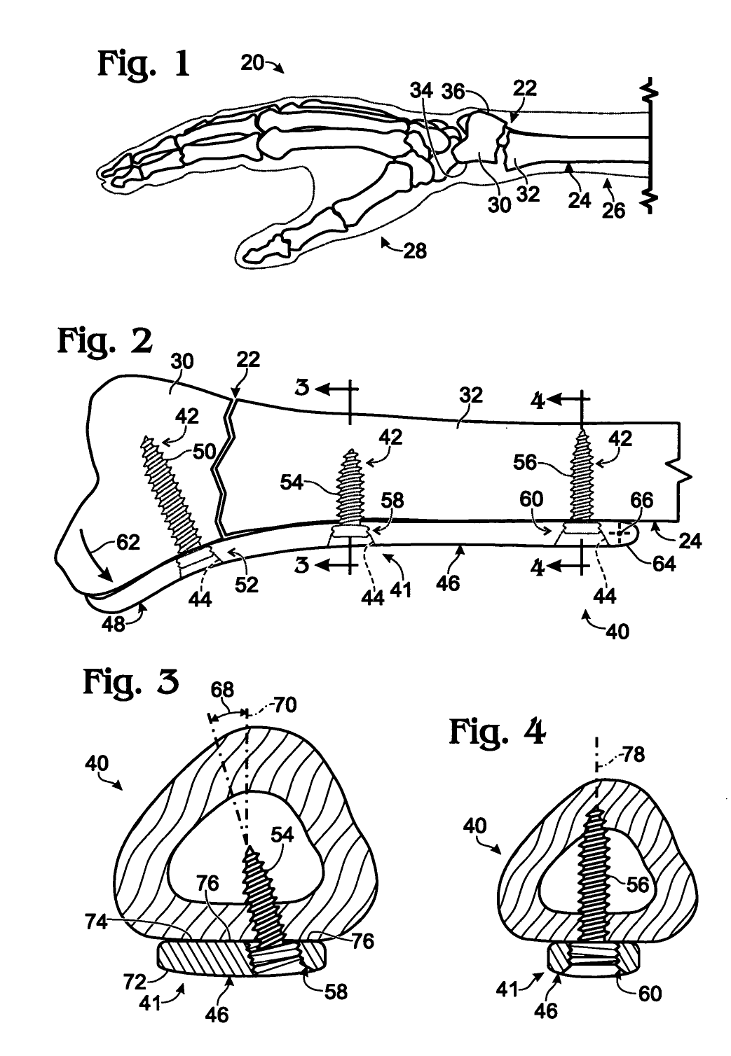

FIG. 1 shows a lateral view of an upper right appendage 20 exhibiting an exemplary distal radius fracture 22. In the present illustration, the fracture is a Colles fracture, a very common fracture of distal radius 24 typically caused by using an outstretched hand to break a fall. The position of the fracture is indicated relative to the skin of the distal forearm 26 and hand 28, which are shown in phantom outline. In Colles fracture 22, a smaller, distal bone fragment 30 (or fragments) may be displaced dorsally from a larger, proximal bone segment 32 of the radius bone. A left or right radius exhibiting Colles fracture 22, or any other suitable fracture or damage, may be fixed with the bone plates described herein.

The bone plates may be configured to be installed on any suitable surface of the distal radius. For example, the bone plates may fit onto the volar (anterior or lower) surface 34 or the dorsal (posterior or upper) surface 36, among others. In some examples, installation on the volar surface may be preferable, because this site of installation may reduce or avoid tendon irritation that may occur with flexion when the bone plate is positioned on the dorsal surface of the radius.

FIG. 2 shows an exemplary system 40 for fixation of fractured distal radius 24. System 40 may include a bone plate 41 secured to the distal radius using locking fasteners 42, such as bone screws. The bone plate may include a plurality of apertures 44 configured to receive the fasteners. Each fastener may be placed into the radius from one of the apertures, as shown here, or may be placed from an opposing direction into one of the apertures from the radius. One or more of apertures 44 may be locking apertures, such as threaded apertures, that engage fasteners to restrict movement of the fasteners in both axial directions.

The bone plate may include a proximal portion 46 configured to be secured generally proximal of fracture 22 (to proximal segment 32), and a distal portion 48 configured to be secured generally distal to fracture 22 (to distal fragment(s) 30). Each of the proximal portion and the distal portion may include one or more locking apertures for receiving a fastener. For example, in the present illustration, a distal bone screw 50 extends into distal fragment 30 from a distal locking aperture, shown at 52. Furthermore, intermediate and proximal bone screws 54, 56 extend into proximal segment 32 from proximal locking apertures, shown at 58, 60, respectively.

One of the challenges of distal radius fixation may be achieving stable attachment of a bone plate to bone. In particular, due to the poor bone quality frequently present in a fractured distal radius, one or more fasteners may tend to pull out of a seated position in bone, in response to forces exerted on the bone plate through the distal radius. These challenges may be compounded by certain fastener placements, which may create lever arms that amplify forces by creating mechanical advantage. For example, a force 62 exerted on distal fragment 30 may urge or pivot proximal portion 46 of the bone plate away from proximal segment 32 of the radius. In particular, a proximal end region 64 of the plate may define a pivot point or pivot axis, shown approximately at 66, about which the force is exerted. One or more fasteners disposed generally centrally along the length of the plate, such as intermediate bone screw 54, thus may play an important role in resisting the force, for example, by reducing the lever arm, to keep the bone plate properly secured to the distal radius. In particular, at least one intermediate fastener may be locked to the bone plate with an offset from orthogonal.

FIGS. 3 and 4 show sectional views of system 40. FIG. 3 shows intermediate bone screw 54 locked to bone plate 41 via locking aperture 58, at an angular offset, shown at 68, from orthogonal, shown at 70. Orthogonal, as used herein, means orthogonal to a plane defined by the bone plate, and particularly a plane defined by the proximal portion of the portion. The plane may be defined by axes along which the length and width of the proximal portion are measured, and/or by a surface of the proximal portion of the plate, among others. For example, proximal portion 46 may include an outer surface 72 that faces away from bone, and an inner surface 74 that faces bone. The outer or inner surface may define the plane. In some embodiments, the plane corresponds to a plane extending tangentially from a surface region 76 adjacent the locking aperture. FIG. 4 shows proximal bone screw 56 locked to bone plate 41 via locking aperture 60 and extending orthogonally, shown at 78, from the proximal portion of the plate. Accordingly, bone screws 54 and 56 may have an angular offset relative to one another. Furthermore, each bone screw may have the same length or may be of different length, as shown in FIGS. 3 and 4, and may be configured to extend unicortically (as with bone screw 54) or bicortically (as with bone screw 56).

A fastener locked to a bone plate at an angular offset may restrict separation of the plate from bone better than an orthogonally directed fastener. In particular, the fastener with the angular offset generally engages a larger cross-sectional area of bone orthogonal to the direction in which the plate is being urged away from bone. Accordingly, the systems of the present teachings may provide more stable attachment of bone plates to the distal radius and/or may permit use of shorter fasteners, to reduce soft tissue irritation.

Further aspects of the present teachings are described in the following sections, including (I) bone plates, (II) fasteners, (III) jigs, (IV) methods of securing bone plates to bones, and (V) examples.

I. Bone Plates

Bone plates of the present teachings generally comprise any plate-like fixation device configured for attachment to bone. The plates may be of a sturdy yet malleable construction. Generally, the plates should be stiffer and stronger than the section of bone spanned by each plate, yet flexible (e.g., springy) enough not to strain the bone significantly. The plates may be unitary, that is, formed as one piece, or may include two or more discrete components. The two or more discrete components may be connected through a mechanical joint that enables translational and/or pivotal movement to adjust the shape and/or size of the bone plates. Further aspects of unitary and multi-component bone plates that are adjustable are described in the patent applications listed above under Cross-References, which are incorporated herein by reference, particularly U.S. patent application Ser. No. 10/716,719, filed Nov. 19, 2003; U.S. patent application Ser. No. 10/717,015, filed Nov. 19, 2003; U.S. Patent Application Serial No. 717,399, filed Nov. 19, 2003; and Ser. No. 10/717,402, filed Nov. 19, 2003.

A. Plate Shape and Structure

The bone plates of the present teachings may have any shape suitable for use on the distal radius. For example, the bone plates may be supplied in precontoured configuration (e.g., by pre-operative bending and/or machining, among others) to include an inner surface that generally matches a surface region of a distal radius, such as a distal volar, distal dorsal, distal lateral, and/or distal medial surface. The bone plates thus may be precontoured according to an average or representative surface geometry of the distal radius. Alternatively, or in addition, the bone plates may be contoured peri-operatively (e.g., by bending), to adjust their shape before and/or during their installation on bone, to improve, for example, the fit of the bone plates on the distal radius for particular individuals. The distal radius, as used herein, refers to any portion of the radius bone that is spaced form the proximal end of the radius bone. Generally, the distal radius refers to the distal about two-thirds, one-half, or one-third of the radius. The bone plates of the present teachings are configured preferably to fix radius bones having fractures or other discontinuities disposed in the distal about one-fourth of the radius, although they may be used more generally to repair any suitable fracture.

Each bone plate may be configured for use on any suitable side or sides of the body. For example, the bone plate may be configured for use on both the left radius and the right radius, such as when the bone plate has general mirror symmetry about its central long axis. Alternatively, each bone plate may be configured for use on either the left radius or the right radius, but not both.

The bone plates may include a proximal portion and a distal portion configured to have a corresponding relative disposition on the distal radius. The proximal portion thus may be configured to be disposed substantially proximal to a bone discontinuity, and the distal portion may be configured to be disposed substantially distal to a bone discontinuity, so that these portions are attached to the radius adjacent opposing sides of the discontinuity.

The proximal and distal portions may be connected to one another through a bridge or junction region of each plate. The junction region may be joined unitarily to each of the proximal and distal portions, to provide a plate member of unitary construction, or may provide a site at which discrete proximal and distal plate components are connected to each other, to provide a plate member of non-unitary construction. The junction region may be configured to allow proximal and distal portions of each plate to slide, bend, turn, and/or twist relative to one another. Alternatively, or in addition, the junction region may provide a site at which a jig may be attached to the plate. Fasteners also or alternatively may be placed into bone from the junction region. However, the junction region may span a discontinuity in bone so that that fastener placement into bone from the junction region may be less desirable that in other portions of the plate.

The bone plates may have any suitable shape defined by the perimeter of the plate. In some examples, the proximal portion may be generally linear, and the distal portion may widen relative to the proximal portion. For example, the plates may be generally T-shaped, with an axially disposed proximal portion and a transversely disposed distal portion, and/or may have a widened fan-like head (the distal portion) connected to an elongate stem (the proximal portion). The width of the proximal portion may be generally constant or more vary along its length. Furthermore, the edges of the proximal portion may be generally linear, curved, and/or sinuous. For example, the width may vary according to the lateral disposition of one or more apertures that are offset and/or staggered in disposition, to produce one or more lateral bulges in the perimeter.

The bone plates may be configured to reduce irritation to the bone and surrounding tissue. For example, the plates may have a low and/or feathered profile to reduce their protrusion into adjacent tissue and rounded, burr-free surfaces to reduce the effects of such protrusion.

The plates may be generally elongate (at least before bending), with a length L, a width W, and a thickness T. Here, length L>width W>thickness T. In use, the long axis of the plates, and particularly of the proximal portion, may be aligned with the long axis of the radius bone and/or may extend obliquely and/or transversely relative to the long axis.

The thickness of the plates generally is defined by a distance between inner (bone-facing) and outer (bone-opposing) surfaces of the plates. The thickness of the plates may vary according to the intended use, for example, to make the plates thinner as they extends over protrusions (such as processes, condyles, tuberosities, and/or the like), reducing their profile and/or rigidity, among others. The thickness of the plates also may be varied to facilitate use, for example, to make the plates thinner, to facilitate bending where they typically need to be contoured peri-operatively. In this way, the plates may be thicker and thus stronger in regions where they typically do not need to be contoured, for example, regions of the plates that are placed along the shaft of the bone, among others. In some examples, the proximal portion of each bone plate may be thicker than the distal portion and/or the bridge region disposed between the proximal and distal portions. A thinner bridge region may permit adjustment of the relative angular disposition of the proximal and distal portions by bending and/or twisting the plate at the bridge region. A thinner distal portion may reduce irritation by reducing the profile of this portion of the plate. In some examples, the proximal and distal portions may have about the same thickness.

B. Plate Apertures

The plates generally include a plurality of apertures (openings) configured to perform similar or different functions. The apertures may be adapted to receive fasteners for affixing the plates to the bone. Alternatively, or in addition, the apertures may be configured to alter the local rigidity of the plate and/or to facilitate blood flow to a fracture or surgical site to promote healing, among others. In some examples, one or more apertures of a plate may be configured for coupling a jig to the bone plate. Each aperture may have any suitable shape, including non-elongate (such as circular) or elongate (such as oval, elliptical, rectangular, etc.). Apertures may be formed and/or tapped (threaded) pre-operatively, such as during the manufacture of the plates, and/or peri-operatively, such as with the plates disposed on bone. Further aspects of tapping apertures peri-operativel are included in U.S. patent application Ser. No. 10/873,410, filed Jun. 21, 2004, which is incorporated herein by reference.

Individual apertures may be locking or nonlocking. Exemplary locking apertures include a thread, ridge, and/or lip for engaging complementary structure on a fastener. The thread and/or the wall of the aperture may be configured to stop over-advancement of a fastener. For example, the thread may terminate in a dead end adjacent the inner surface of the plate, and/or the thread or aperture may taper inward toward the inner surface. Alternatively, or in addition, structure to stop over-advancement of the fastener may be included in the fastener as described below in Section II. Other locking apertures are described in the patent applications listed above under Cross-References, which are incorporated herein by reference, particularly U.S. Provisional Patent Application Ser. No. 60/548,685, filed Feb. 26, 2004.

The plates may include one or more locking apertures offset from orthogonal in the proximal portion of each plate. Each locking aperture defines an axis (the aperture axis) corresponding to the long axis of a fastener locked into the aperture. Offset from orthogonal, as used herein, means that the aperture axis forms a nonzero angle with an axis extending orthogonally (as defined elsewhere in the present teachings) to the proximal portion. Accordingly, an aperture defining an aperture axis with an angular offset is configured to lock a fastener in a disposition such that the fastener extends nonorthogonally from the proximal portion. In some examples, the nonzero angle defined by the aperture axis may be at least about five degrees, at least about ten degrees, or about five to twenty degrees. In exemplary embodiments, the nonzero angle is about ten degrees. The aperture axis may be related to an orthogonal axis defined by the proximal portion by any suitable direction of rotation. For example, the aperture axis may be rotated from orthogonal about an axis parallel to a central long axis of the proximal portion and/or about an axis parallel to a transverse axis of the proximal portion. In some examples, the proximal portion may include at least a pair of locking apertures, each with an angular offset. The angular offset may be of the same or different magnitude, and in the same or different directions of rotation. For example, the pair of locking apertures may define axes having opposite directions of rotation from orthogonal. Alternatively, or in addition, the pair of locking apertures may be offset laterally (translationally) to opposing sides of a central long axis defined by the proximal portion of the plate. In some embodiments, the proximal portion may include three or more locking apertures disposed on alternating sides of a center line of the proximal portion.

The bone plates may have one or more openings configured as slots. A slot is any opening having a length that is greater than its width. The slot may be linear, arcuate, or angled, among others. The slot may include a counterbore structure to receive a head of a bone screw. The counterbore structure may be configured, as in a compression slot, to exert a force generally parallel to the long axis of the slot when a bone screw is advanced against the counterbore structure. Slots may extend axially, that is, in general alignment with the long axis of the plate, or transversely, that is, substantially nonparallel to the long axis, that is, oblique to the long axis or orthogonal to the long axis. Each bone plate may have one or more axial slots and one or more transverse slots. The slots may be used to adjust the translational and/or angular disposition of each bone plate one bone. Further aspects of slots that may be included in the bone plates of the present teachings are described further in the patent applications listed above under Cross-References, which are incorporated herein by reference, particularly, U.S. patent application Ser. No. 10/717,015, filed Nov. 19, 2003.

The bone plates may be configured to receive wires. Each bone plate thus may include one or more holes extending through the plate between inner and outer surfaces of the plate. Alternatively, or in addition, the bone plates may be configured to receive and retain wires that extend over the plates, rather than through the plates, from bone spaced from the plates. Further aspects of bone plates configured to secure wires are described in the patent applications listed above under Cross-References, which are incorporated herein by reference, particularly U.S. Provisional Patent Application Ser. No. 60/563,767, filed Apr. 19, 2004.

C. Plate Materials

The bone plates may be formed of any suitable biocompatible material(s). Accordingly, the bone plates may be formed of, or include, metal, polymer, plastic, ceramic, composite, and/or the like. Exemplary biocompatible materials for forming bone plates may include metals/metal alloys (for example, titanium or titanium alloys; alloys with cobalt, chromium, and/or molybdenum; stainless steel; etc.) and/or bioresorbable materials (such as polygalactic acid (PGA), polylactic acid (PLA), polycaprolactones, polydioxanones, copolymers thereof, etc.), among others.

Further aspects of bone plates that may be suitable for use in the bone plates of the present teachings are described in the patent applications listed above under Cross-References, which are incorporated herein by reference, particularly Ser. No. 10/716,719, filed Nov. 19, 2003; Ser. No. 10/717,015, filed Nov. 19, 2003; Serial No. 717,399, filed Nov. 19, 2003; Ser. No. 10/717,402, filed Nov. 19, 2003; and Ser. No. 10/731,173, filed Dec. 8, 2003.

II. Fasteners

The fasteners generally comprise any mechanism for affixing a bone plate to a bone. Exemplary fasteners may include bone screws, pins, and wires, among others.

A bone screw generally includes any fastener with a threaded shank configured for placement into bone. The bone screw may be a locking screw or a nonlocking screw. A locking screw, as used herein, is any screw configured to engage an aperture (generally a wall thereof) of a bone plate so that axial movement of the locking screw relative to the bone plate is restricted in both axial directions (defined by the screw) by engagement with the bone plate. The locking screw may have a thread configured to threadably engage the aperture and/or may have any other projection(s) or depression(s) that allow the screw to lock to the bone plate. Any suitable combination of locking and nonlocking bone screws each may be placed into locking and/or nonlocking apertures of a bone plate.

Any suitable portion of the locking screw may engage the aperture, to lock the screw to the plate, based, for example, on how the locking screw approaches the bone plate. In some examples, a head of the locking screw (e.g., a thread formed on the head) may engage a wall of the aperture (e.g., a thread formed on the wall), to lock the screw in position, such as when the locking screw is placed first through the aperture and then into bone. The head may be nontapered or may be tapered. For example, the head may taper toward the shank of the screw, to restrict over-advancement of the head into or through the aperture. In some examples, a distal region of the screw's shank (e.g., a thread formed distally on the shank) may lock the screw in position (e.g., through engagement with a thread formed on the aperture wall), such as when the locking screw is placed first through bone and then into the aperture. The distal region of the shank may be configured to restrict over-advancement of the distal region into or past the aperture, for example, by having a thread that dead-ends proximally and/or by including a shoulder proximal to the thread, so that the shoulder engages the inner surface of the bone plate and restricts further advancement.

A locking or nonlocking bone screw may have any suitable length. The length may be sufficient to penetrate the bone cortex once (a unicortical screw), adjacent the bone plate. Alternatively, the length may be sufficient to penetrate the bone cortex twice (a bicortical screw), adjacent and opposite the plate. Generally, unicortical screws provide less support than bicortical screws, because they penetrate less cortex. Unicortical and bicortical bone screws may have relatively small threads for use in hard bone, such as typically found in the shaft portion of a bone. Alternatively, or in addition, the bone screws may be cancellous bone screws having relatively larger threads for use in soft bone, such as typically found near the ends (peri-articular regions) of a long bone. The arrangement of openings described herein may enable the use of shorter or unicortical screws in one or more apertures of the bone plates and/or may enable one-screws (unicortical or bicortical) to function more effectively in holding the plate on bone.

Wires may be placed into any suitable number of apertures of a bone plate. Each wire may act to provisionally secure the bone plate to bone, or may serve to secure smaller fragments that cannot be secured with screws or pins. In some examples, wires may be placed into larger apertures, for example, when the wires are of larger diameter or when the wires are used to guide drilling with a cannulated drill bit to form a hole for a screw or pin, among others.

Pins may be placed into any suitable number of apertures of a bone plate. In some examples, the pins may be configured to include a threaded head and a substantially nonthreaded shank. Accordingly, the pins may be used in place of bone screws in one or more locking apertures of the bone plate.

Fasteners may have any other suitable features. For example, the fasteners may be self-drilling and/or self-tapping, so that they form their own holes or threads as they are advanced into bone.

Further aspects of fasteners that may be suitable are described in the patents and patent applications listed above under Cross-References, which are incorporated herein by reference, particularly U.S. Pat. No. 6,030,162, issued Dec. 18, 1998; U.S. Pat. No. 6,299,615, filed Aug. 16, 1999; U.S. Provisional Patent Application Ser. No. 60/548,685, filed Feb. 26, 2004; and U.S. patent application Ser. No. 10/873,410, filed Jun. 21, 2004.

III. Jigs

The systems of the present teachings may include a jig to facilitate securing bone plates to bone with fasteners. A jig, as used herein, is any device that guides a tool and/or a fastener during installation of a bone plate on bone. The tool or tools guided by the jig may include a drill (particularly a drill bit thereof), a reamer, and/or a driver for advancing fasteners. The fasteners guided by a jig may include any of the fasteners described above in Section II.

The jig may be positioned relative to the bone plate by any suitable mechanism. For example, the jig may connect to the bone plate and/or to bone adjacent the bone plate. If connected to the bone plate, the jig may use any suitable connection. In some embodiments, the jig may use one or more apertures of the bone plate for connection. The one or more apertures may be threaded or nonthreaded. Alternatively, or in addition, the jig may engage an edge of the bone plate. The jig may include an inner surface having a contour that substantially matches a portion (or all) of the outer surface of the bone plate. In some examples, the jig may be a cannula that threads into an aperture of the bone plate through which the jig will guide a tool or fastener. Alternatively, the jig may include a plurality of channels configured for concurrent alignment with each of a plurality of apertures in the bone plate.

Further aspects of jigs and methods of using jigs are described below in Example 3 and in the patent applications listed above under Cross-References, which are incorporated herein by reference, particularly U.S. patent application Ser. No. 10/717,401, filed Nov. 19, 2003; and U.S. Provisional Patent Application Ser. No. 60/563,860, filed Apr. 19, 2004.

IV. Methods of Securing Bone Plates to Bones

The systems of the present teachings include methods of securing bone plates to bones. Exemplary steps that may be included in the methods are described below. These steps may be performed in any suitable order, in any suitable combination, and each step may be performed any suitable number of times.

A bone for fixation may be selected. The bone may be either a right or a left radius bone in a person of any suitable age, size, gender, etc. The bone may be fractured by one or more fractures, may be cut, may have a malunion or nonunion, and/or may be otherwise structurally unsound.

The bone may be placed in a condition for fixation. For example, the bone may be exposed by surgical procedure (such as creating an incision in soft tissue overlying the bone) and any fractures of the bone may be reduced.

A bone plate with an offset aperture may be selected for installation on the bone. The bone plate may be selected according to the handedness (right or left side of body) of the bone, size of bone, severity of injury (such as size, number, and/or position of fracture(s)), surface onto which the plate will be secured, and/or the like. Selection of a bone plate thus may include selecting a right- or left-sided bone plate, and/or a bone plate with a particular configuration of its proximal portion and/or apertures, from a larger set of available bone plates (a kit). In some examples, bone plates of the set may include indicia (such as alphanumeric characters, a color, a barcode, and/or another marking) formed on their surface to facilitate identification of particular plate configurations. The selected bone plate may be used directly or the shape of the bone plate may be adjusted peri-operatively (e.g., with a tool and/or with the hands) to better match a surface geometry of the selected bone. The bone plate may be sterile before installation, for example, contained in a sterilized package.

The bone plate may be secured to the bone using suitable fasteners, such as screws, pins, and/or wires. The bone plate may be fastened to any suitable surface of the bone. However, preferably the surface is a distal surface, for example, a distal volar or distal dorsal surface of the radius. If the bone includes a discontinuity, proximal and distal portions of the bone plate may be secured to the bone adjacent opposing sides of the discontinuity.

Fasteners may be placed into apertures of the bone plate in any suitable order, and in either direction. For example, one or more fasteners may be placed first into the proximal portion of the bone plate, to secure the proximal portion to a proximal segment of bone. At least one of the fasteners placed into the proximal portion may be locked to the bone plate at an angular offset from orthogonal to the proximal portion. Furthermore, one or more fasteners may be placed into the distal portion of the bone plate and into bone after (or before) the proximal portion is secured to the bone. Each fastener introduced into an aperture of the distal portion may be locked or not locked to the distal portion. In some examples, a surgeon may select from a locking fastener or a nonlocking fastener for placement into a locking aperture, so that the fastener either locks to the plate or does not lock to the plate. In addition, the surgeon may select from a nonlocking fastener that fits more tightly and is held at a substantially fixed angle in the locking aperture, or a nonlocking fastener that fits less tightly and thus can be placed into bone at various angles from the locking aperture. In some examples, fasteners may be locked to the distal portion from opposing directions of approach to the plate.

A jig (or jigs) may be used to guide formation of holes in bone and/or placement of fasteners. In some examples, the jig may be used to drill holes into bone and/or place fasteners into bone from generally opposing sides of the bone.

Further aspects of securing bone plates to the distal radius and use of jigs or related guide systems for forming holes and/or placing fasteners are included in the patent applications listed above under Cross-References, which are incorporated herein by reference, particularly U.S. patent application Ser. No. 10/716,719, filed Nov. 19, 2003; U.S. patent application Ser. No. 10/717,015, filed Nov. 19, 2003; U.S. Patent Application Serial No. 717,399, filed Nov. 19, 2003; U.S. patent application Ser. No. 10/717,401, filed Nov. 19, 2003; U.S. patent application Ser. No. 10/717,402, filed Nov. 19, 2003; and U.S. Provisional Patent Application Ser. No. 60/563,860, filed Apr. 19, 2004.

### V. EXAMPLES

The following examples describe selected aspects and embodiments of exemplary systems for distal radius fixation. These selected aspects and embodiments include bone plates with angularly offset apertures, a jig for use with the bone plates, and methods of using the bone plates and jig, among others. These examples are included for illustration and are not intended to limit or define the entire scope of the present teachings.

### Example 1

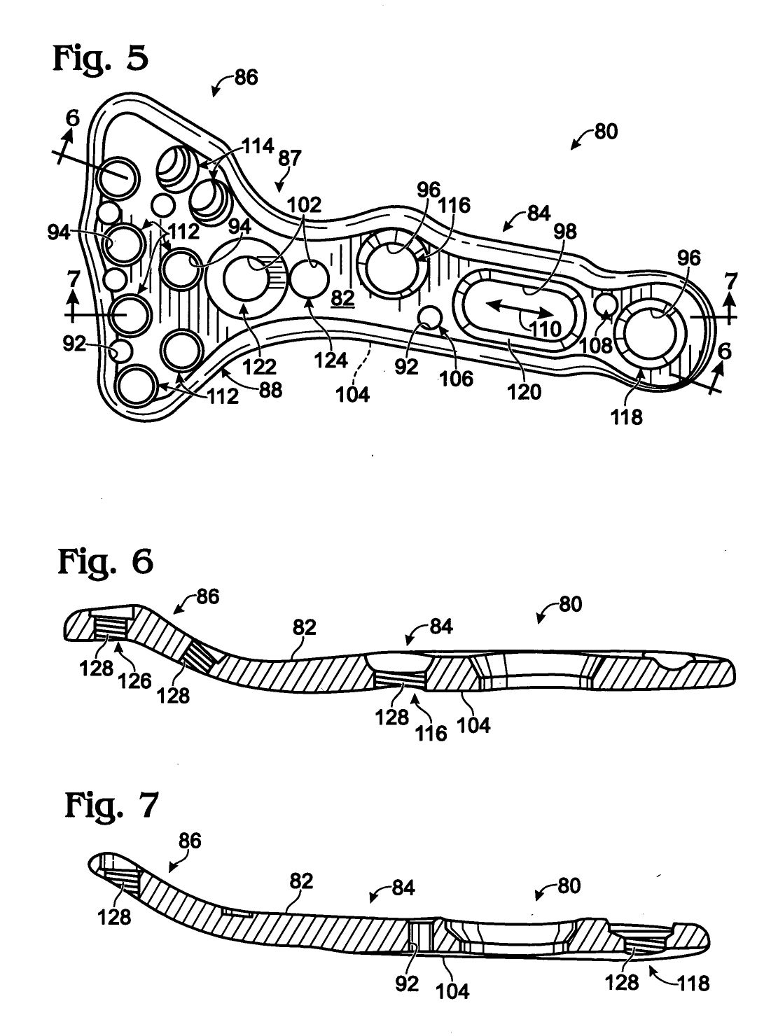

This example describes an exemplary bone plate 80 configured for distal radius fixation and including an angularly offset aperture; see FIGS. 5-9.

FIG. 5 shows bone plate 80 as viewed from above outer surface 82 of the plate. Plate 80 may include a proximal portion 84, a distal portion 86, and a bridge region 87 disposed between the proximal and distal portions. The distal portion may be wider than the proximal portion and may taper toward the proximal portion, shown at 88.

Plate 80 may include a plurality of apertures that extend into and/or through the plate in each portion of the plate. Each of the apertures may be locking (e.g., threaded) or nonlocking (nonthreaded). The apertures may include smaller, generally nonthreaded through-holes 92 for receiving wires (six are shown here), locking (threaded) distal apertures 94 (eight are shown here) and locking (threaded) proximal apertures 96 (two are shown here) for receiving threaded fasteners, one or more elongate openings, such as oval-shaped slot 98, and/or coupling apertures 102 (two are shown here) for securing a jig to the plate.

One or a plurality of through-holes 92 for wires may be disposed in each portion of the plate. Each through-hole may extend orthogonally through the plate, between outer surface 82 and inner surface 104, or may extend with an angular offset from orthogonal. In some examples, the proximal portion may include a pair of through-holes, such as holes shown at 106, 108, that each have an angular offset from orthogonal and from each other. The angular offset may correspond to rotation about an axis substantially parallel to central long axis or center line 110 of the proximal portion, and may be in the same rotational direction, or in opposite rotational directions, so that wires placed through the holes extend toward (and/or through) a longitudinally disposed central plane extending orthogonally from the proximal portion of the bone plate.

One or more of distal apertures 94 may be angularly offset from one another and/or from the bone plate. For example, in the present illustration, parallel apertures shown at 112 (six are shown here) are configured to engage threaded fasteners such that the fasteners extend along substantially parallel paths in bone. Nonparallel apertures shown at 114 are configured to engage threaded fasteners such that the fasteners extend nonparallel to fasteners locked to parallel apertures 112 and nonparallel with one another. Nonparallel apertures 114 may be configured to engage and lock bone screws that extend into (or from) the radial styloid region of the radius.

One or more of proximal apertures 96 may be angularly offset. For example, the offset aperture shown at 116 may be configured to lock a bone screw at an offset from orthogonal to the proximal portion, and particularly orthogonal to a plane defined by inner surface 104 of the proximal portion (or an inner surface region thereof). Offset aperture 116 also may be offset laterally from central long axis 110 of the proximal portion. When laterally offset, offset aperture 116 may be configured to direct a locked fastener generally toward a cross-sectional center of the radius, or away from this center. Proximal-end aperture, shown at 118, also may be a locking aperture, and may direct a locked fastener along a path orthogonal to the proximal portion (or with an angular offset).

Slot 98 may be disposed generally between offset aperture 116 and proximal-end aperture 118. The slot may extend substantially axially along proximal portion 84, that is, parallel to central long axis 110. Slot 98 may be configured to permit a fastener placed into bone to slide axially along the plate (and/or the plate to slide axially relative to the fastener and bone). This sliding movement may be used to adjust the position of the bone plate on bone and to compress pieces of the bone axially. Accordingly, slot 98 may include a counterbore 120 that forms a lip with varying thickness along its length, to create a compression slot.

Jig-coupling apertures 102 may include a locking aperture, shown at 122, and an orientation aperture, shown at 124. Locking aperture 122 may be threaded, to receive and threadably engage a coupling member extending through the jig. Orientation aperture 124 may be configured to receive a pin or boss of the jig, to restrict rotation of the jig about the coupling member. Further aspects of an exemplary jig and coupling the jig to bone plate 80 are described below in Example 3.

FIGS. 6 and 7 show longitudinal sectional views of bone plate 80. Plate 80 may have a generally planar proximal portion 84, to fit onto a generally planar region of the volar surface of the distal radius, such that the proximal portion appears linear in a longitudinal sectional view. The plate also may bend out of a plane defined by the proximal portion, as it extends into distal portion 86, to fit onto a corresponding concave volar surface region of the distal radius. The sectional profile of the distal portion may vary transversely, to match the transversely varying shape of the volar surface of the distal radius. In particular, the lateral side of the distal portion may form an oblique or transverse groove 126, to fit onto a corresponding ridge present on the volar surface of the distal radius.

FIGS. 6 and 7 also show additional aspects of plate 80. For example, each locking aperture may have a thread 128. Other additional aspects of the bone plate, such as hole 92, surfaces 82, 104, and apertures 116, 118 are numbered for clarity and to facilitate comparison with FIG. 5.

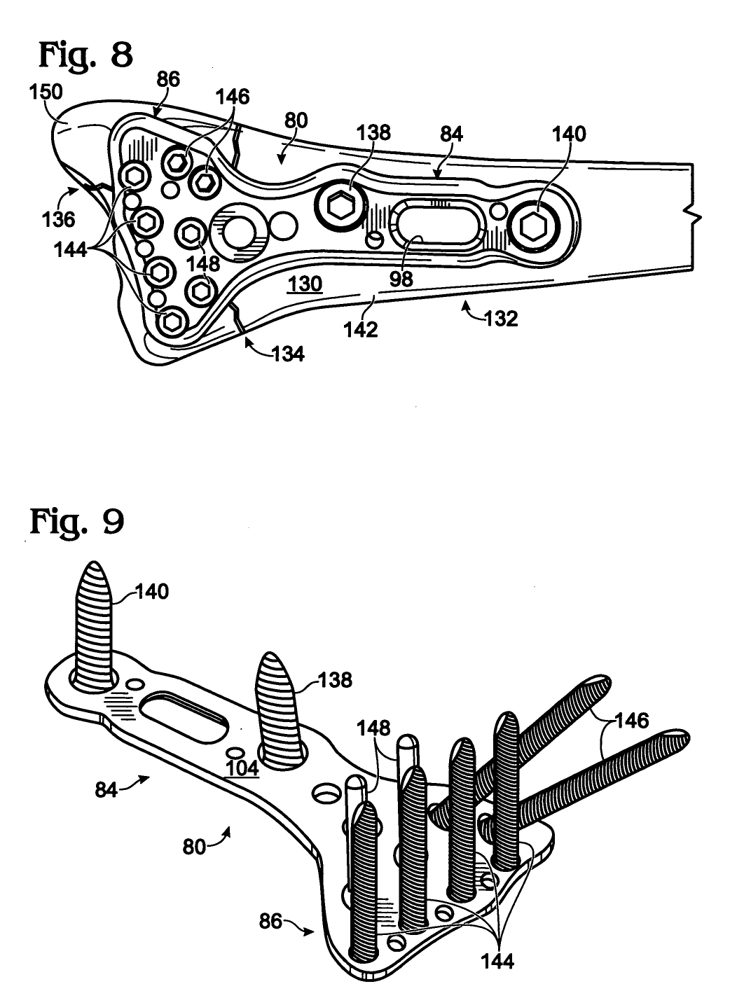

FIG. 8 shows bone plate 80 secured to a volar surface 130 of a distal region of a right-sided radius bone 132. The radius bone has suffered a transverse fracture 134 and a longitudinal fracture 136, to break the radius into three pieces. However, bone plate 80 may be used to fix a radius broken into fewer or more pieces. Proximal bone screws 138, 140 secure proximal portion 84 to a proximal segment 142 of the radius, and distal bone screws 144, 146 and pins 148 secure distal portion 86 of the plate to distal fragments of the radius. For example, non parallel bone screws 146 extend into styloid process 150 of the radius. In the present illustration, slot 98 has not received a bone screw. In other configurations, any suitable other subset (or all) of the apertures may have received a bone screw. For example, only a subset of the threaded apertures in the distal portion may receive bone screws and the remainder may be left unoccupied.

FIG. 9 shows bone plate 80 and the fasteners of FIG. 8, as viewed from the inner surface of the bone plate, in the absence of the fractured distal radius. Proximal screws 138, 140 each may be locked to the plate. Proximal-end screw 140 may extend orthogonally to the proximal portion of the plate, particularly a proximal region of inner surface 104, and intermediate screw 138 may extend with an angular offset to the proximal portion and to the proximal-end screw. In particular, the intermediate screw may be offset by rotation about an axis parallel to the long axis of the proximal portion. Distal bone screws 144 and pins 148 may be locked to the bone plate so that they extend parallel to each other from the inner surface of the plate. Styloid screws 146 also may be locked to the plate, but may extend nonparallel to the other fasteners and to each other. In other configurations, any other suitable combination of pins and screws may be locked to any suitable set of distal apertures.

### Example 2

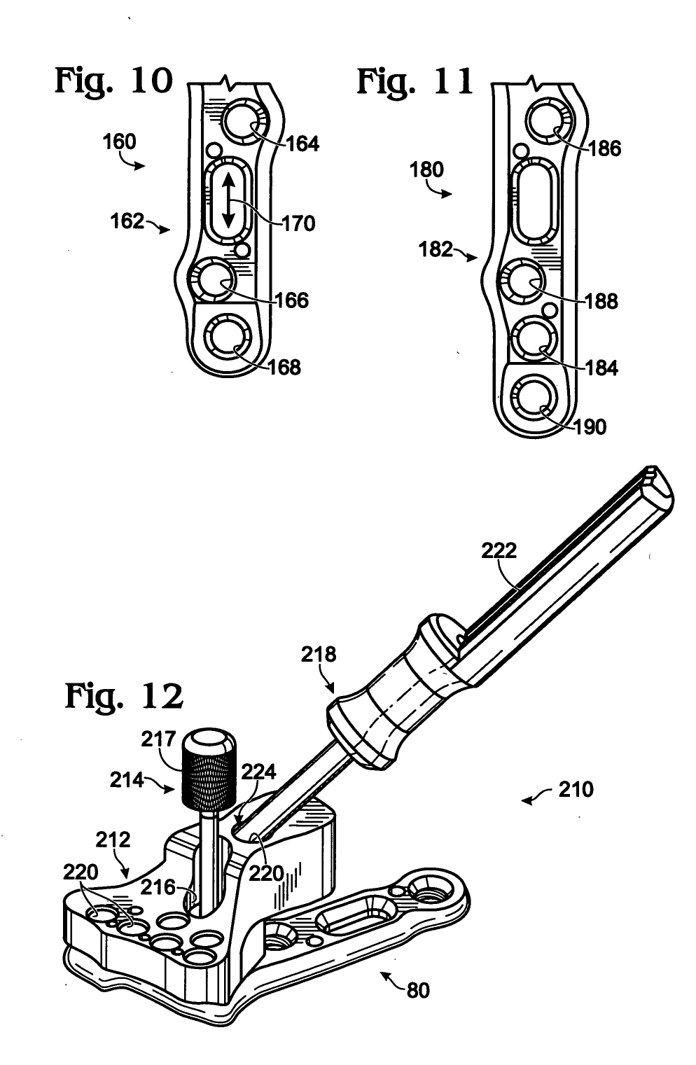

This example describes alternative proximal portions that may be included in the bone plate of Example 1; see FIGS. 10 and 11.

FIG. 10 shows a fragmentary view of an exemplary bone plate 160 having a proximal portion 162 of greater length than the proximal portion of bone plate 80 and a distal portion corresponding to the distal portion of bone plate 80 (see FIGS. 5-9). Proximal portion 162 may include a pair of offset locking apertures 164, 166, each disposed distally of a proximal-end aperture 168. Locking apertures 164, 166 may be threaded and may define axes (and thus paths along which locked fasteners extend) that are offset from orthogonal to the bone plate, and angularly offset from one another. The locking apertures also may be laterally offset from a central axis 170 of the bone plate, to opposing sides of the central axis. The axes defined by the locking apertures (and thus fasteners locked to these apertures) may extend nonparallel to one another and may be angled inward, that is, each intersecting an orthogonal plane (including the central axis and extending orthogonally from the plate) at a position spaced from the inner surface and closer to the inner surface than the outer surface.

FIG. 11 shows a fragmentary view of another exemplary bone plate 180 having a proximal portion 182 of greater length than the proximal portion of bone plate 80 and a distal portion corresponding to the distal portion of bone plate 80 (see FIGS. 5-9). Proximal portion 182 may include an additional locking aperture 184, relative to proximal portion 162 of bone plate 160 (see FIG. 10). Additional locking aperture 184 may be configured to lock a fastener orthogonal to (or offset from orthogonal to) the proximal portion of the bone plate and may be disposed between a set of offset apertures 186, 188 and a proximal-end aperture 190.

### Example 3

This example describes an exemplary system to guide formation of holes in bone and/or placement of fasteners into the bone, to facilitate and/or effect attachment of a bone plate to bone; see FIGS. 12-14.

FIG. 12 shows a system 210 including a jig 212 secured to bone plate 80 (see FIGS. 5-9). System 210 also may include a coupling member 214 that extends through an opening 216 in the jig, to thread into a coupling aperture 122 (see FIG. 5) of plate 80, to hold the jig on the bone plate. Coupling member may include a handle 217 configured to be rotated by hand or may include tool-engagement structure. System 210 further may include a guide member, such as a cannulated drill guide 218, received removably in guide channels 220 of the jig. Drill guide 218 may define a passageway 222 configured to receive a drill bit for drilling into bone. In the present illustration, the guide member is placed in one of two alternative channels of a bifurcated channel, shown at 224, that extends to distal apertures 114 (see FIG. 5).

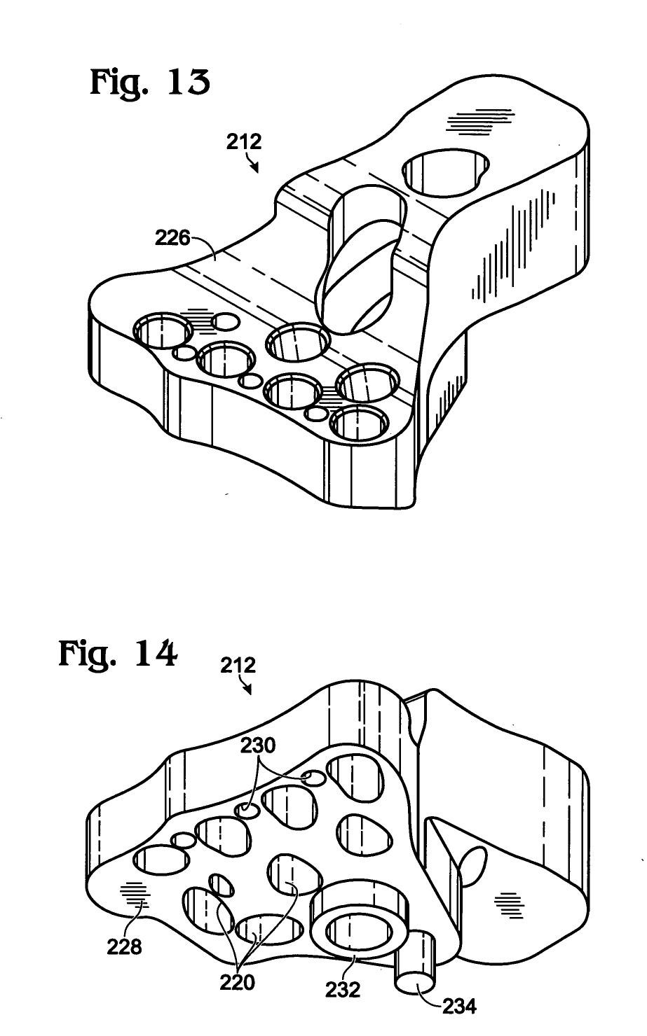

FIGS. 13 and 14, respectively, show jig 212 from above an outer surface 226 and below an inner surface 228 of the jig. Guide channels 220 may extend between the outer surface and the inner surface of the jig and in alignment with each of the distal apertures of plate 80 (see FIG. 5). The guide channels may be sized to receive cannulated drill guide 218 (see FIG. 12) for drilling holes into bone and/or to guide a fastener into bone. Some of the guide channels, such as smaller holes 230, may be sized for placement of wires through corresponding holes in the bone plate. The jig may have a footprint corresponding to some or all of the distal portion of the bone plate.

The inner surface of jig 212 may be configured for mating with bone plate 80. In particular, the inner surface may include an annular ridge 232 that extends from opening 216 of the jig (see FIG. 12) and is sized to fit into coupling aperture 122 of bone plate 80 (see FIG. 5). Furthermore, the inner surface may include a boss or pin 234 configured to be received in orientation aperture 124 of bone plate 80 (see FIG. 5).

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in applications claiming priority from this or a related application. Such claims, whether directed to a different invention or to the same invention, and whether broader, narrower, equal, or different in scope to the original claims, also are regarded as included within the subject matter of the inventions of the present disclosure.

1. A bone plate for fixation of distal radius fractures, comprising:
a plate member including a proximal portion and a distal portion and having a plurality of apertures, the proximal portion and distal portion being configured to be secured to a distal surface region of a radius bone adjacent opposing sides of a fracture in the radius bone using fasteners received in the plurality of apertures, the proximal portion including a locking aperture configured to receive a fastener that locks to the proximal portion such that the fastener extends at an angular offset from orthogonal to the proximal portion.

2. The bone plate of claim 1, wherein the proximal portion has an inner surface configured to face the radius bone, wherein a region of the inner surface is disposed adjacent the locking aperture, and wherein orthogonal to the proximal portion is defined as orthogonal to a plane tangential to the region of the inner surface.

3. The bone plate of claim 1, the locking aperture being a first aperture having a thread, wherein the proximal portion includes a second aperture having a thread and being disposed more proximally than the first aperture.

4. The bone plate of claim 3, the fastener being a first fastener, wherein the second aperture is configured to receive a second fastener threadably, such that the second fastener extends at least substantially orthogonal to the proximal portion.

5. The bone plate of claim 1, wherein the proximal portion has a proximal end and a length measured from the proximal end to the distal portion of the plate member, and wherein the locking aperture is spaced from the proximal end of the proximal portion by at least about one-half of the length of the proximal portion.

6. The bone plate of claim 1, the proximal portion defining a long axis, wherein the angular offset corresponds to rotation of the fastener about an axis substantially parallel to the long axis.

7. The bone plate of claim 1, wherein the proximal portion includes two or more locking apertures each configured to receive a fastener that locks to the proximal portion such that each fastener extends at an angular offset from orthogonal to the proximal portion.

8. The bone plate of claim 7, wherein at least a pair of the two or more locking apertures are offset in opposing rotational directions.

9. The bone plate of claim 1, wherein the proximal portion includes at least one aperture that is nonthreaded.

10. The bone plate of claim 1, wherein the plate member is configured to be secured to a volar surface region of the radius bone.

11. The bone plate of claim 1, wherein the distal portion of the plate member is wider than the proximal portion.

12. The bone plate of claim 1, wherein the plate member has a unitary structure.

13. A bone plate for fixation of distal radius fractures, comprising:
a plate member configured to fix a radius bone and having a plurality of apertures configured to receive fasteners that secure the plate member to a distal region of the radius bone, the plate member including a proximal portion defining a plane and a distal portion extending out of the plane, the proximal portion including a locking aperture configured to receive a fastener that locks to the plate member such that the fastener extends at an angular offset from orthogonal to the plane.

14. The bone plate of claim 13, wherein the proximal portion includes an inner surface, and wherein the inner surface defines the plane.

15. The bone plate of claim 13, wherein the proximal portion defines a center line, and wherein the angular offset corresponds to rotation about an axis parallel to the center line.

16. The bone plate of claim 13, wherein the locking aperture includes a thread.

17. The bone plate of claim 13, the locking aperture being a first locking aperture, wherein the proximal portion includes a second locking aperture configured to receive a fastener that locks to the plate member.

18. A method of fixing distal radius fractures, comprising:
selecting a bone plate configured to be installed on a distal surface region of a radius bone, the bone plate including a proximal portion and a distal portion;
selecting a radius bone having a fracture disposed distally;
securing the proximal portion and distal portion to the radius bone such that the proximal and distal portions are disposed adjacent opposing sides of the fracture,
wherein securing includes locking a fastener to an aperture of the proximal portion such that the fastener extends at an angular offset from orthogonal to the proximal portion.

19. The method of claim 18, wherein the step of securing installs the bone plate on a volar surface region of the radius bone.

20. The method of claim 18, the fastener being a first fastener and the aperture being a first aperture, wherein the step of securing includes locking a second fastener to a second aperture of the proximal portion, before or after locking the first fastener to the first aperture.
